(19) United States
(12) Patent Application Publication
(10) Pub. No.: US 2019/0269774 A1
(43) Pub. Date: Sep. 5, 2019

(54) LIQUID ALLERGEN COMPOSITIONS AND METHODS FOR MAKING THE SAME

(71) Applicant: BEFORE BRANDS, Inc., Menlo Park, CA (US)

(72) Inventors: Ashley Dombkowski, Woodside, CA (US); Christopher Cornyn, Woodside, CA (US); Albert E. Rosevear, Jr., Ithaca, NY (US); Olivia M. Weihe, Ithaca, NY (US); Cosmin M. Beliciu, Ithaca, NY (US); Alexandra F. Russell, Ithaca, NY (US)

(21) Appl. No.: 16/349,440

(22) PCT Filed: Nov. 21, 2017
(86) PCT No.: PCT/US2017/062781

§ 371 Date: May 13, 2019

Related U.S. Application Data
(60) Provisional application No. 62/424,854, filed on Nov. 21, 2016.

(51) Int. Cl. A61K 39/35; A61K 9/00; A61K 9/14; A61K 9/107; A23L 33/125; A23L 33/155; A23L 29/269

(52) U.S. Cl. CPC A61K 39/35; A61K 9/0095; A61K 9/14; A61K 2039/542; A23L 33/125; A23L 33/155; A23L 29/272; A61K 9/107

(57) ABSTRACT

Liquid mixed allergen compositions of two or more different allergens are provided. Also provided are methods of making liquid mixed allergen compositions and administering a liquid mixed allergen composition to a subject.

### CROSS-REFERENCE TO RELATED APPLICATIONS

This application claims the benefit of and priority to U.S. application Ser. No. 62/424,854 filed Nov. 21, 2016, which is hereby incorporated by reference in its entirety.

### BACKGROUND

Allergy is a disorder of the immune system characterized by the occurrence of allergic reactions to normally harmless environmental substances. Allergies are caused by allergens, which may be present in a wide variety of sources, including but not limited to pollens or other plant components, dust, molds or fungi, foods, additives, latex, transfusion reactions, animal or bird danders, insect venoms, radiocontrast medium, medications or chemicals. Common allergic reactions include eczema, hives, hay fever, asthma, and reactions to venoms. Mild allergies like hay fever are highly prevalent in the human population and cause symptoms such as allergic conjunctivitis, itchiness, and runny nose. In some people, severe allergies to environmental or dietary allergens or to medication may result in life-threatening anaphylactic reactions and potentially death, if left untreated.

A food allergy is an adverse immune response to a food allergen, e.g., a food protein. Common food allergens are found in shellfish, peanuts, tree nuts, fish, milk, eggs, soy and fresh fruits such as strawberries, mango, banana, and apple. Immunoglobulin E (IgE)-mediated food allergies are classified as type-I immediate hypersensitivity reactions. These allergic reactions have an acute onset (from seconds to one hour) and the accompanying symptoms may include angioedema (soft tissue swelling of the eyelids, face, lips, tongue, larynx and trachea), hives, itching of the mouth, throat, eyes, or skin, gastrointestinal symptoms such as nausea, vomiting, diarrhea, stomach cramps, or abdominal pain, rhinorrhea or nasal congestion, wheezing, shortness of breath, or difficulty swallowing, and even anaphylaxis, a severe, whole-body allergic reaction that can result in death. It is estimated that 1 out of 12 children under the age of 21 years of age have a doctor's diagnosis of food allergies, and over $24 billion is spent per year on health care costs for food allergic reactions, largely due to about 90,000 visits to the ER per year in the U.S. due to food induced anaphylactic symptoms. Moreover, there are still deaths that occur every year due to fatal food allergies.

Accordingly, there exists a need in the art for compositions that can prevent and/or treat allergies, and processes for making such compositions.

### SUMMARY

The disclosure is directed, at least in part, to a process for producing a homogenized liquid mixed allergen composition. For example, this disclosure provides for a process for producing a homogenized liquid mixed allergen composition comprising: dry blending a dry mixture, wherein the dry mixture comprises 6 or more allergens, e.g., 6 to 20 allergens, and a bulking agent; milling the dry mixture and passing the milled dry mixture through a large screen (e.g., with an opening size of about 0.033 inches) to obtain a first pass mixture; milling the first pass mixture and passing the first pass mixture through a small screen (e.g., with an opening size of about 0.020 inches) to obtain a fine particle mixture with substantially consistent particle size; mixing the fine particle mixture into water (e.g., shear mixing) to obtain a hydrated mixture; and passing the hydrated mixture through a homogenizer to obtain a homogenized liquid mixed allergen composition. Milling the dry mixture may include using a rotor speed of about 7,500 RPM, for example, milling the first pass mixture may use a rotor speed of about 7,500 RPM.

As part of a contemplated process, milling the dry mixture and/or milling the first pass mixture may further comprise pulsing a vacuum suction through the mill.

Dry blending a dry mixture as part of a contemplated process may include, for example, dry blending a dry mixture comprising 6 or more allergens, e.g., 6 to 20 allergens, and a bulking agent comprising maltodextrin and/or sucrose. In an embodiment, a contemplated bulking agent comprises maltodextrin and sucrose in a weight ratio of about 3:1 maltodextrin to sucrose. In an alternative embodiment, one or more additional allergens are dry milled separately and optionally mixed with the dry mixture, the first pass mixture, and/or the fine particle mixture.

A contemplated process may further comprise hydrating the fine particle mixture in the water for about 1 hour or more and/or processing the liquid mixed allergen mixture at an ultra-high temperature (e.g., about 287° F. or higher). In some embodiments, processing the liquid mixed allergen mixture at an ultra-high temperature occurs after passing the hydrated mixture through the homogenizer.

In an exemplary embodiment, a contemplated process further comprises shear mixing the homogenized liquid allergen mixture with one or more excipients to obtain a shear-mixed homogenized liquid allergen mixture. For example, such excipients may each be selected from the group consisting of: a food safe oil, a polysaccharide (e.g., gellan gum), flavoring, and a food safe salt (e.g., dipotassium phosphate).

A contemplated process, may, in some embodiments, further comprise in-line homogenizing the shear-mixed homogenized liquid allergen mixture. In some embodiments, in-line homogenizing the shear-mixed homogenized liquid allergen mixture occurs after processing at an ultra-high temperature.

Also contemplated herein is a liquid mixed allergen composition comprising a homogenized liquid mixed allergen composition wherein the homogenized liquid mixed allergen composition is produced by a disclosed process.

For example, provided herein is a liquid homogenized mixed allergen composition comprising: 12 to 16 different protein allergens; maltodextrin; sucrose; oil, and optionally a vitamin (e.g., vitamin D), wherein the weight ratio of maltodextrin to sucrose is about 3:1. Disclosed liquid homogenized mixed allergen compositions may further comprise gellan gum.

In another embodiment, provided herein is a unit dose of about 20 mL to about 30 mL of a liquid homogenized mixed allergen composition comprising: 12 to 16 different protein allergens; maltodextrin; sucrose; oil, and optionally a vitamin, wherein the weight ratio of maltodextrin to sucrose is about 3:1.

### BRIEF DESCRIPTION OF THE DRAWINGS

FIG. 1 depicts a comparison of a pre-milled and post-milled dry mixed allergen composition.

### DETAILED DESCRIPTION

Disclosed herein are liquid mixed allergen compositions of two or more different allergens, and processes for making such compositions.

### Processes of Making Mixed Allergen Compositions

Provided herein is a process for producing a homogenized liquid mixed allergen composition, e.g., a liquid mixed allergen composition such as described herein. A disclosed process may, e.g., comprise one or more of the following steps: dry blending a dry mixture, wherein the dry mixture comprises 6 to 20 allergens and a bulking agent; milling the dry mixture and passing the milled dry mixture through a large screen to obtain a first pass mixture; milling the first pass mixture and passing the first pass mixture through a small screen to obtain a fine particle mixture with substantially consistent particle size; mixing the fine particle mixture into water to obtain a hydrated mixture; and/or passing the hydrated mixture through a homogenizer to obtain a homogenized liquid mixed allergen composition.

In certain embodiments, a disclosed process comprises dry blending a dry mixture of allergens, e.g., an allergen described herein, and a bulking agent, e.g., a bulking agent described herein. The dry mixture of allergens may include any allergen or allergen composition described herein. In certain embodiments, a mixture of allergens may comprise one, two, or more allergens each independently selected from the allergens disclosed in the Examples herein. For example, in certain embodiments, a composition may comprise one, two, or more allergens selected from a group consisting of peanut, soy, almond, cashew, hazelnut, pecan, pistachio, walnut, wheat, oat, milk, egg, cod, salmon, shrimp, and sesame. In certain embodiments, the dry mixture of allergens includes about 30 mg each of peanut, soy, almond, cashew, hazelnut, pecan, pistachio, walnut, wheat, oat, milk, egg, cod, salmon, shrimp, and sesame. It will be appreciated that the allergens contemplated herein may each be present as a meal, flour, and/or powder, and at least some of allergens are contemplated as initially in dry form.

Contemplated bulking agents may include any bulking agent described herein. In certain embodiments, the bulking agent comprises maltodextrin, sucrose, or a combination of maltodextrin and sucrose, e.g., maltodextrin and sucrose at a weight ratio of about 3:1. Without wishing to be bound by theory, it is believed that bulking agents reduce the fat content of an allergen mixture to aid in downstream processing, e.g., milling.

In certain embodiments, a disclosed process comprises milling the dry mixture. The milling may, e.g., comprise using a rotor speed of about 7,500 RPM, or may, e.g., further comprise pulsing a vacuum suction through the mill. In certain embodiments, a disclosed process comprises milling a first pass mixture. The milling may, e.g., comprise using a rotor speed of about 7,500 RPM, or may, e.g., further comprise pulsing a vacuum suction through the mill. Without wishing to be bound by theory, it is believed that milling reduces grittiness and large particle size, allowing for even distribution throughout a liquid composition.

In certain embodiments, a disclosed process comprises milling a dry mixture and passing the milled dry mixture through a large screen to obtain a first pass mixture; and milling the first pass mixture and passing the first pass mixture through a small screen to obtain a fine particle mixture. The large screen may, e.g., have an opening size of about 0.033 inches. The large screen filter may, e.g., have an opening size of about 0.020 inches.

In certain embodiments, a disclosed process comprises mixing of a fine particle mixture into water. In certain embodiments, the mixing of a fine particle mixture into water comprises shear mixing. The fine particle mixture may, e.g., be further hydrated in water for about 1 hour or more following shear mixing.

In certain embodiments, a disclosed process comprises passing a hydrated mixture through a homogenizer to obtain a homogenized liquid mixed allergen. The homogenizer may be run, e.g., at about 6,000 to about 7,000 psi. The hydrated mixture may be passed through the homogenizer one time or more than one time, e.g., two times, three times, or more than three times. Without wishing to be bound by theory, it is believed that passing a hydrated mixture through a homogenizer reduces particle size and results in a more consistent range of suspending particles in the hydrated mixture.

In certain embodiments, a disclosed process further comprises processing a liquid mixed allergen mixture at an ultra-high temperature, e.g., about 287° F. or higher. Processing the liquid allergen mixture at the ultra-high temperature may, e.g., occur after passing the hydrated mixture through a homogenizer. Without wishing to be bound by theory, it is believed that processing at an ultra-high temperature ensures safety of the composition.

In certain embodiments, a disclosed process further comprises shear mixing a homogenized liquid allergen mixture with one or more excipients to obtain a shear-mixed homogenized liquid allergen mixture. The excipients may comprise any excipient described herein. The one or more excipients may, e.g., each be selected from the group consisting of: a food safe oil, a polysaccharide (e.g., gellan gum), flavoring, and a food safe salt (e.g., dipotassium phosphate). In certain embodiments, a disclosed process further comprises in-line homogenizing the shear-mixed homogenized liquid allergen mixture.

As used herein, a “mixed allergen composition” is understood to mean a composition that includes two or more different allergens, where any two given allergens are different if they are distinct from each other, e.g., they are compounds described by different chemical formula or compositions described by different components and/or amounts thereof. The number of different allergens in a composition may vary, as desired. In certain embodiments, a mixed allergen composition comprises 2 or more different allergens, such as 3 or more different allergens, 4 or more different allergens, 5 or more different allergens, 6 or more different allergens, 7 or more different allergens, 8 or more different allergens, 9 or more different allergens, 10 or more different allergens, 15 or more different allergens, 20 or more different allergens, 25 or more different allergens, 30 or more different allergens, 40 or more different allergens, 50 or more different allergens, 75 or more different allergens, or 100 or more different allergens. In certain embodiments, a mixed allergen composition comprises 100 or fewer different allergens, such as 75 or fewer different allergens, 50 or fewer different allergens, 25 or fewer different allergens, 15 or fewer different allergens, or 10 or fewer different allergens. In certain embodiments, a composition may include 2 to 20 different allergens, 2 to 100 different allergens, or 2 to 1000 different allergens. In further embodiments, a composition may comprise 6 to 20 different allergens. In certain embodiments, a composition may consist essentially of 6 to 20 different protein allergens.

Allergens present in the composition may vary, where in some instances an allergen present in the composition is one that induces an allergy in a susceptible subject. Allergens include any antigen, or active derivative thereof, that elicits a specific IgE response. Antigens include any substance that can stimulate the production of antibodies and can combine specifically with them. Allergens may have little or no intrinsic toxicity by themselves, but cause a pathological condition due to their ability to elicit an IgE-associated immune response, and, upon subsequent exposure, due to their ability to elicit IgE- and/or T cell-dependent hypersensitivity reactions. As such, an allergen includes any substance which is capable of stimulating a typical hypersensitivity reaction in atopic subjects. Allergens that may be present in a given mixed allergen composition include any substance found in a variety of different sources, e.g., foods, drugs, perfume, plants, the environment or biological systems (e.g., prokaryotic or eukaryotic cells or viruses), as well as chemical allergens.

It is appreciated that reference to an allergen or an allergen composition (e.g., such as part of a provided food product or composition) may each include a plurality of different proteins as found in the naturally occurring allergen (either raw or cooked). For example, a provided food product may include a peanut allergen composition (which would include substantially all peanut proteins present in e.g., defatted peanuts, ground peanuts, etc.). As used herein the phrase “complete allergen” refers to all possible antigenic components of a given food product.

Allergens of interest include nut allergens. Nut allergens are allergens that include one or more compounds found in nuts, e.g., dry fruits that include an edible kernel or meat enclosed in a woody or leathery shell. Nut allergens of interest include, e.g. peanut allergens, (e.g., rAra h 1, rAra h 2, rAra h 3, rAra h 8 PR-10, rAra h 9 LTP, or peanut complete allergen), brazil nut allergens (e.g., rBer e 1, or brazil nut complete allergen), hazelnut or filbert allergens (e.g., rCor a 1 PR-10, rCor a 8 LTP, nCor a 9, rCor a 14, or hazelnut complete allergen), walnut allergens (e.g., rJug r 1, rJug r 3 LTP, or walnut complete allergen), cashew allergens (e.g., cashew component allergens, or cashew complete allergen), pistachio allergens (e.g., pistachio component allergens, or pistachio complete allergen), pecan allergens (e.g., pecan component allergens, or pecan complete allergen), almond allergens (e.g., almond component allergens, or almond complete allergen), or tree nut component package allergens (e.g., one or more allergens from e.g., cashew nut, walnut, hazelnut, or brazil nut).

Allergens of interest include animal allergens. Animal allergens are allergens that include one or more compounds found in animals, including both vertebrates and invertebrates. Vertebrate animal allergens that may be present in a mixed allergen composition include avian allergens (e.g., egg allergens, e.g., nGal d 1 Ovomucoid, n Gal d 2 Ovalbumin, nGal d 3 Conalbumin, or egg white complete allergen), mammalian allergens (e.g. milk allergens, e.g., nBos d 4 alpha-lactalbumin, nBos d 5 beta-lactoglobulin, nBos d 8 Casein, nBos d Lactoferrin, or milk complete allergen), or fish allergens (e.g., rCyp c 1, rGad c 1, cod complete allergen, white fish allergens, or pink fish allergens). Invertebrate animal allergens that may be present in a mixed allergen composition include crustacean allergens (e.g., shrimp allergens, e.g., rPen a 1 tropomyosin, or shrimp complete allergen), or insect allergens (e.g., bee sting venom allergen, wasp sting venom allergen, or mosquito bite allergen).

Allergens of interest include non-nut plant allergens, i.e., plant allergens that are not nut allergens. Plant allergens are allergens that include one or more compounds found in plants. Plant allergens of interest include wheat allergens (e.g., rTri a 19 Omega-5 Gliadin, gliadin wheat, rTri a 14 LTP, or wheat complete allergen), fruit allergens (e.g., kiwi allergens, e.g., rAct d 8 PR-10, or kiwi complete allergen), vegetable allergens (e.g., carrot allergens, or celery allergens, e.g., rApi g 1.01 PR-10, rPhl p 12, or celery complete allergen), CCD MUXF3 from Bromelain, legume allergens (e.g., soy allergens or chickpea allergens, e.g., rGly m 4 PR-10, nGly m 5 Beta-conglycinin, nGly m 6 Glycinin, or soy complete allergen), stone fruit allergens (e.g., f419, f420, f421, f95, f242, o214 rPru p 1 PR-10, rPru p 3 LTP, or stone fruit primary complete allergen), oat allergens (e.g., oat component allergens, or oat complete allergen), or seed allergens (e.g., sesame allergens, e.g., sesame seed component allergens, or sesame see complete allergen).

Additional types of allergens that may be present in mixed allergen compositions include, e.g., non-food animal allergens (e.g., cat or dog fur and dander, cockroach calyx, dust mite excretion), drug allergens (penicillin, sulfonamides, salicylates, local anesthetics), mold spore allergens, latex allergens, metal allergens, or plant pollen allergens (e.g. from grass, e.g., ryegrass or timothy-grass, from weeds, e.g., ragweed, plantago, nettle, Artemisia, vulgaris, Chenopodium album, sorrel, or e.g., from trees, e.g., birch alder, hazel, hornbeam, aesculus, willow, poplar, platanus, tilia, or olea).

In certain embodiments, a composition may comprise one, two, or more allergens selected from a group consisting of cashew, pistachio, walnut, pecan, white fish, pink fish, shrimp, peanut, soy, hazelnut, almond, milk, egg, crab, wheat, and sesame.

In certain embodiments, a composition may comprise one, two, or more allergens each independently selected from the allergens disclosed in the Examples herein. For example, in certain embodiments, a composition may comprise one, two, or more allergens selected from a group consisting of peanut, soy, almond, cashew, hazelnut, pecan, pistachio, walnut, wheat, oat, milk, egg, cod, salmon, shrimp, and sesame.

The amount of a given allergen in a mixed allergen composition may vary, as desired. In certain embodiments, the amount of a given allergen ranges from about 1 mg to about 15,000 mg, about 5 mg to about 15,000 mg, about 10 mg to about 10,000 mg, about 15 mg to about 5,000 mg, about 10 mg to about 100 mg, or about 15 mg to about 100 mg. In certain embodiments, the amount of a given allergen is about 30 mg. The weight percentage of a given allergen in a mixed allergen composition may vary, as desired. In certain embodiments, the weight percentage of a given allergen in a mixed allergen composition ranges from about 0.1 wt. % to about 99.9 wt. %, about 0.1 wt. % to about 15 wt. %, about 0.1 wt. % to about 99.9 wt. %, about 15 wt. % to about 99.9 wt. %, or about 25 wt. % to about 65 wt. %. The amount of a given allergen in a mixed allergen composition may be recited by total mass, or by protein mass, which may vary for a given allergen depending upon the weight percentage of protein in that allergen.

In certain embodiments, any two of the mixed allergens, or all of the mixed allergens, are present in equal parts, e.g., in a 1:1 ratio, such that each allergen is present in the composition in equal weight.

Disclosed mixed allergen compositions may include one or more vitamins, as desired. Vitamins that may be present in the compositions include. e.g., vitamin A (e.g., in an amount ranging from 1 to 35,000 IU), vitamin C (e.g., in an amount ranging from about 1 to about 1,000 mg), vitamin D (e.g., in an amount ranging from about 1 to about 4,000 IU, i.e., from about 0.025 to about 100 mcg), vitamin E (e.g., in an amount ranging from about 1 to about 450 IU) vitamin K (e.g., in an amount ranging from about 1 to about 250 mcg), vitamin B-1 (thiamin; e.g., in amount ranging from about 1 to about 15 mg), vitamin B-2 (riboflavin; e.g., in an amount ranging from about 1 to about 17 mg) vitamin B-3 (niacin; e.g., in an amount ranging from about 1 to about 200 mg), vitamin B-5 (pantothenic acid; e.g., in an amount ranging from about 1 to about 100 mg), vitamin B-6 (pyridoxine; e.g., in an amount ranging from about 1 to about 30 mg) vitamin B-9 (folic acid; e.g., in an amount ranging from about 1 to about 4,000 mcg), vitamin B-12 (cobalamin; e.g., in an amount ranging from about 1 to about 250 mcg), vitamin H (biotin; e.g., in an amount ranging from about 1 to about 1,000 mcg) and combinations thereof. In certain embodiments, a mixed allergen composition comprises vitamin D. In certain embodiments, a mixed allergen composition comprises about 400 IU, i.e., about 10 mcg, of vitamin D.

Also provided are physiological acceptable compositions that include a disclosed mixed allergen composition and a physiologically acceptable delivery vehicle. Disclosed mixed allergen compositions can be incorporated into a variety of formulations for administration to a subject. More particularly, a disclosed mixed allergen composition can be formulated into physiological acceptable compositions by combination with appropriate, physiologically acceptable carriers or diluents. In certain embodiments, a disclosed mixed allergen composition is designed for oral administration, for example, as foods, tablets, troches, lozenges, aqueous or oily suspensions, dispersible powders or granules, emulsions, hard or soft capsules, or syrups or elixirs, gums, etc. Compositions intended for oral use may be prepared according to any convenient protocol for the manufacture of pharmaceutical compositions and such compositions may contain one or more agents selected from the group consisting of sweetening agents, flavoring agents, coloring agents and preserving agents in order to provide palatable preparations.

In certain embodiments the disclosure provides for a process of making a liquid composition that is a food product. Food products of interest include a disclosed mixed allergen composition in combination with a food delivery vehicle. By food delivery vehicle is meant a delivery vehicle that is a nourishing substance that is eaten, drunk, or otherwise taken into the body to sustain life, provide energy, promote growth, etc. Examples of food delivery vehicles or food products of interest include, but are not limited to: baby or infant formula, baby food (e.g., pureed food suitable for infant or toddler consumption), chips, cookies, breads, spreads, creams, yogurts, liquid drinks, chocolate containing products, candies, ice creams, cereals, coffees, pureed food products, etc. In certain embodiments, the composition is a food supplement.

In certain embodiments, a disclosed mixed allergen composition is in a liquid form. In certain embodiments, a liquid mixed allergen composition may include a bulking agent. Exemplary bulking agents include maltodextrin, sucrose, trehalose, trehalose dihydrate, mannitol, lactose, or raffinose or any combination thereof. In certain embodiments, the bulking agent comprises maltodextrin, or sucrose, or a combination thereof. In certain embodiments, the bulking agent comprises maltodextrin and sucrose at a weight ratio of about 3:1. In certain embodiments, a liquid mixed allergen composition may include excipients, e.g., a food safe oil, a polysaccharide (e.g., gellan gum), flavoring, and a food safe salt (e.g., dipotassium phosphate).

In certain embodiments a mixed allergen composition is an aqueous suspension containing a disclosed mixed allergen component in admixture with excipients suitable for the manufacture of aqueous suspensions. Such excipients may include suspending agents, for example sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

In certain embodiments a mixed allergen composition is an oily suspension containing a mixed allergen composition suspended in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Disclosed physiologically acceptable compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, preservative and flavoring and coloring agents. A disclosed composition may be in the form of a sterile aqueous or oleagenous suspension. This suspension may be formulated according to the known art using those suitable dispersing or wetting agents and suspending agents which have been mentioned above. The sterile preparation may also be a sterile solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

A mixed allergen composition may be a unit dose composition, by which is meant that it is present in a composition that is configured to be administered to a subject as a single dose, which single dose may or may not be part of a dosing schedule made up of two or more unit dosages that are administered to a subject over a given a period of time. A disclosed unit dosage may be recited by mass or volume. In certain embodiments, a unit dose may have a mass ranging from about 300 mg to about 20 grams, such as about 300 mg, about 400 mg, about 500 mg, about 600 mg, about 700 mg, about 800 mg, about 900 mg, about 1000 mg (1 g), about 1.5 g, about 2 g, about 3 g, about 4 g, about 5 g, about 10 g, about 15 g, or about 20 g. In certain embodiments, a unit dose has a mass of about 480 mg. In certain embodiments, a unit dose may have a volume ranging from about 20 mL to about 30 mL, such as about 20 mL, about 21 mL, about 22 mL, about 23 mL, about 24 mL, about 25 mL, about 26 mL, about 27 mL, about 28 mL, about 29 mL, or about 30 mL.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Throughout the description, where apparatus, devices, and systems are described as having, including, or comprising specific components, or where processes and methods are described as having, including, or comprising specific steps, it is contemplated that, additionally, there are apparatus, devices, and systems that consist essentially of, or consist of, the recited components, and that there are processes and methods that consist essentially of, or consist of, the recited processing steps.

The foregoing examples are presented herein for illustrative purposes only, and should not be construed as limiting in any way.

### EXAMPLES

### Example 1

This example describes the selection of ingredients for inclusion an exemplary liquid mixed allergen composition containing 16 allergenic ingredients (almond, cashew, cod, egg, hazelnut, milk, oat, peanut, pecan, pistachio, salmon, sesame, shrimp, soy, walnut, and wheat)

First, ingredients were sourced with primary emphasis on domestic commercial viability, with exceptions made for ingredients that were only available internationally. Successful commercial sourcing of multiple options per each allergenic ingredient led to the development of selection criteria in order to choose the best commercial ingredient to be tested. Attributes screened included maximum protein content and minimum bulking materials, organoleptic attributes, including overall taste, presence of off-notes, grittiness, and ingredient solubility. Ingredients of considerably low protein content, or with large proportions of bulking ingredients, were eliminated from contention. By group consensus, ingredients were tasted dry to determine the presence of off-flavors, as well as assess the ingredient's grittiness.

The allergenicity of selected ingredients was also confirmed. Two initially selected ingredients, salmon protein and wheat protein, failed protein gels for allergen confirmation. It is hypothesized that since these proteins undergo partial or full hydrolysis in their ingredient processing, the amino acid structure is disrupted such that the polypeptide profile does not result in allergenicity. Despite the processability and organoleptic advantages of these ingredients, salmon powder and wheat gluten powder from different sources replaced the wheat and salmon ingredients in the allergenic ingredient blend. The salmon powder is not pure salmon; it is blended with cod protein in order to be milled into a dry ingredient in a ratio of approximately 3:1 salmon to cod. As such, the salmon ingredient percent in the final formula is increased to over-deliver on salmon, in order to ensure adequate delivery of protein from this specific allergen.

### Example 2

This example describes the determination of dry milling procedures in the preparation of an exemplary liquid mixed allergen composition containing 16 allergenic ingredients (almond, cashew, cod, egg, hazelnut, milk, oat, peanut, pecan, pistachio, salmon, sesame, shrimp, soy, walnut, and wheat).

Dry milling the allergenic ingredients was required in order to incorporate the ingredients into a successful liquid supplement format, as many of the ingredients exhibited grittiness and large particle size in their inherent state. Particularly, the nut ingredients (pecan, pistachio, almond), exhibited a wide range of particle sizes. Without dry milling, these ingredients would not be distributed evenly throughout a liquid composition, have the potential to limit processability during downstream steps, affect filling into and out of a package, and will likely settle out over the supplement's shelf life.

Unless indicated otherwise, milling was conducted as follows. The 16 protein ingredients were weighed proportionally in order to deliver 30 mg of each protein and dry blended bulking ingredients. Milling was performed with a Quadro Laboratory Scale Dry Mill (FitzMill L1A) at rotor speeds ranging from 5,000 to 9,000 RPM, and screen sizes of 0.020″, 0.033″, or 0.065″. The ingredient was passed through the mill once (with a single screen), or multiple times (with increasingly smaller screens). Where indicated, continuous or pulsing vacuum suction was applied to pull product through the mill.

A limitation of dry milling is the fat content of the dry blend. Dry blends with fat content above 6% are not able to be milled without considerable processing issues, as the fat seeps from the initial ingredients and obstructs the milling sieve, thus halting the dry milling process altogether. The blend of the 16 allergenic ingredients alone exceeded 38% fat, so a large amount of bulking material was added in order to feasibly mill the blend. Two bulking materials were explored: maltodextrin and a maltodextrin—sugar blend (3:1 ratio). The maltodextrin—sugar blend yielded the best results. The sugar helped sweeten the beverage overall, and it is hypothesized that the crystalline structure of the sugar helped break apart large particles in the dry blend in conjunction with the blades of the mill.

Several other milling variables were explored in order to run product through the mill without clogging, specifically, mill rotor speed, screen size, and the use of vacuum suction to pull product through the mill. A moderately high mill speed was selected to achieve smaller particle size, as the high rotor speeds result in a smaller range of fine particles. Running the mill rotor at capacity, 9,000 RPM, had the risk of product buildup within the mill. If product was allowed to remain within the mill too long, the friction of the mill blades against the product and sieve would result in fat leeching from the particles and sugars caramelizing onto the sieve, effectively blocking the sieve from allowing particles through. As such, mill rotor speed was reduced to 7,500 RPM, which achieved a favorable particle size without product buildup. In addition, vacuum suction aided in pulling the product through the mill. By pulsing the vacuum, the product was able to run through the sieve at a moderate pace and clear the chamber. Continuous vacuum sometimes exhibited too much pull on the product, forcing it against the sieve and blocking the sieve before particles could be ground down to a size that would pass through the sieve. Lastly, two separate passes through the dry mill were required to achieve the best particle size, both organoleptically and from a processability standpoint. Given the range of particle sizes in the 16 allergenic ingredient blend with bulking materials, an initial pass through a larger screen opening size, 0.033″, followed by a smaller screen opening size, 0.020″, yielded a consistent particle size without clogging the mill during the process.

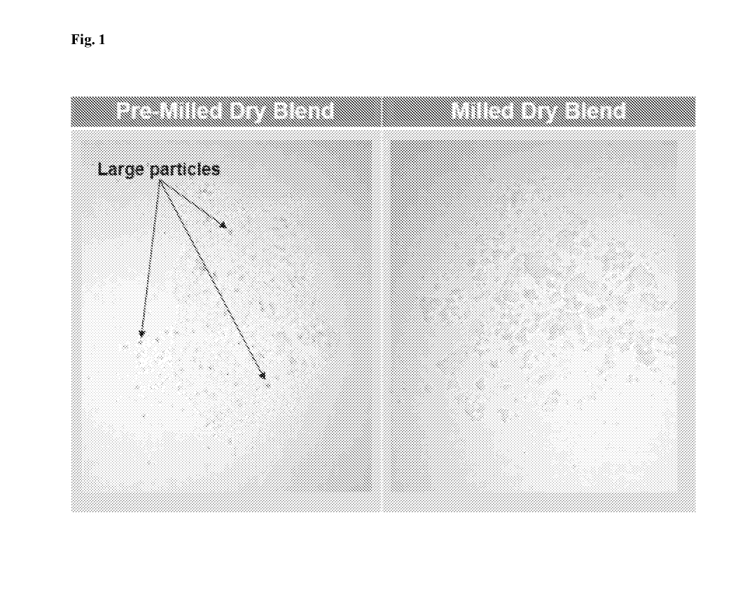

FIG. 1 compares the pre-milled product against the successfully milled dry blend. Large dark particulates were clearly visible in the pre-milled blend, but were no longer apparent after milling. These particles are likely from the nut meals, as these were the grittiest ingredients. In addition, the overall color of the milled product was darker than the pre-milled product. In milling, fat was released from high fat ingredients and distributed throughout all of the dries, which darkened the color slightly. Darkening may also be caused by caramelization due to friction and heat generation during milling. Overall a tight range of finer particles was achieved with the two stage dry milling procedure, as opposed to the pre-milled dry blend.

### Example 3

This example describes the determination of homogenization procedures in the preparation of an exemplary liquid mixed allergen composition containing 16 allergenic ingredients (almond, cashew, cod, egg, hazelnut, milk, oat, peanut, pecan, pistachio, salmon, sesame, shrimp, soy, walnut, and wheat).

Similar to the goal of dry milling, homogenizing was explored to further reduce particle size of solids in the liquid matrix. Dry milling prior to homogenization was necessary to get particles within a range to be further reduced, and to feed the homogenizer with product that had a more consistent range of particles suspended within it.

Dry milled product was shear mixed into water using a Robot Coupe Shear Mixer (MP 350 Turbo) and allowed to hydrate for one hour to allow proteins to hydrate fully. Homogenizers used in the preparation of the composition included an APV Gaulin Laboratory Homogenizer (Model 15 MR), and a GEA Niro Soavi S.p.A. In-Line Homogenizer (Type NS2006H). For initial testing the APV was run between approximately 6,000 and 7,000 psi. Product was run through the homogenizer 1 to 3 times.

Like running the dry mill at high rotor speeds, the homogenizer was run at high pressure in order to have a large reduction in particle size. Homogenizer variables (one, two and three passes through the homogenizer) were compared against a raw un-homogenized sample. While all samples exhibited some settling out over time, the sample passed through the homogenizer three times showed the greatest product opacity and the least amount of sedimentation. The opacity indicates that the fat is better distributed throughout the aqueous matrix in smaller globules, and therefore the product is more stable and consistent.

### Example 4

This example describes the determination of a target volume of an exemplary liquid mixed allergen composition containing 16 allergenic ingredients (almond, cashew, cod, egg, hazelnut, milk, oat, peanut, pecan, pistachio, salmon, sesame, shrimp, soy, walnut, and wheat) for use as a supplement in baby food.

Liquid addition of a placeholder protein beverage to various baby foods provided a working range to target the supplement serving size. Foods tested included Premier Protein Vanilla Shake, ¾ Fresh Banana (an equivalent portion to a serving of Gerber Baby Foods Banana, 2^{nd} foods), and Gerber Baby Foods, including Apple Blueberry, 2^{nd} Foods, Banana, 1^{st} Foods, Chicken Noodle, 2^{nd} Foods, and Roasted Vegetable & Chicken, 3^{rd} Foods

There was not a large difference between the 10 mL and 20 mL additions for most foods; however, the progression from 20 mL to 30 mL was more noticeable. In general, this trend was more prominent in less viscous products, such as applesauce, as less viscous products flow fairly readily. For example, the partially mashed fresh banana exhibited larger changes when the protein beverage was added. This increase in change perception may be due to the fact that the banana was not completely mashed homogenously, compared to processed baby food. Nonetheless, the fresh banana product was still deemed consumable at 30 mL. In contrast, both the Roasted Vegetable & Chicken and the Chicken Noodle processed baby foods held up well to 30 mL addition of the protein beverage, as these blended products are inherently thick.

Given the amount of bulking ingredients needed to add to the 16 allergenic ingredient blend, the dose of the dry blend alone exceeds 9 grams. As such, the minimum volume was estimated to be about 20 mL, as this serving results in a beverage with approximately 40% solids. Incorporating the learnings from the baby food mix-in experiment results in a target serving size range of 20 to 30 mL, with the preference being to achieve the smallest serving size that is processable.

### Example 5

This example describes the determination of a complete manufacturing process for an exemplary liquid mixed allergen composition containing 16 allergenic ingredients (almond, cashew, cod, egg, hazelnut, milk, oat, peanut, pecan, pistachio, salmon, sesame, shrimp, soy, walnut, wheat) for use as a supplement in baby food.

Results obtained from Examples 1-4 were used in the design of trial manufacturing processes for an exemplary liquid mixed allergen composition containing 16 allergenic ingredients (almond, cashew, cod, egg, hazelnut, milk, oat, peanut, pecan, pistachio, salmon, sesame, shrimp, soy, walnut, and wheat).

A process flow for a tested manufacturing process was as follows. Except where indicated, all process steps were conducted as described previously. First, in a dry mill, the 16 allergenic ingredients in proper ratios for protein dosing along with bulking ingredients to lower moisture and fat content to acceptable levels (maltodextrin and sugar) were dry milled through 0.033″ and then 0.020″ particle screens to achieve desired particle size. Next, the dry milled mix was incorporated into liquid format by shear mixing the milled dry mix into lukewarm water to solubilize and suspend the solids, and the product was held for 1 hour following shear mixing to fully hydrate the proteins. Next, the shear mixed product was homogenized to reduce particle size in the liquid suspension or slurry, with multiple homogenizer passes required for decreased sedimentation and improved opacity. Following homogenization, other ingredients were incorporated by shear mixing in fat, buffer, and gellan gum to improve stability, visual appearance and the suspension of the solids. Flavor was added, if needed. Next, an ultra-high temperature unit (MicroThermics UHT/HTST Lab, Model 25 HV Hybrid) was used for ultra-high temperature processing for 15 seconds at 287° F. for a thermal lethality step to ensure food product safety. The resulting product was further homogenized with an in-line homogenizer to produce consistent range of particle sizes for solid components and fat globules, and to uniformly distribute particles throughout the liquid matrix. Lastly, the composition was clean filled into sanitized bottles for refrigerated storage. An aseptic fill would allow for a shelf-stable product.

Pilot plant trials incorporated learnings and processes from the dry mill, pre-U.H.T. homogenization and target volume experiments to initially produce two variables for assessment. Table 1 compares the formulations of these two variables. For the initial trials, two different concentrations were explored to confirm processability, 25 g serving size (Protocept A) and 20 g serving size (Protocept B).

TABLE 1

| | % Total Formula | |
|---|---|---|
| Ingredient | Protocept A | Protocept B |
| Peanut Flour | 0.25 | 0.31 |
| Soy Protein Isolate | 0.14 | 0.17 |
| Blanched Almond Flour | 0.56 | 0.70 |
| Cashew Flour | 0.67 | 0.83 |
| Hazelnut Flour | 0.41 | 0.52 |
| Pecan Meal | 1.33 | 1.67 |
| Pistachio Meal | 0.57 | 0.71 |
| Walnut Flour | 0.28 | 0.35 |
| Wheat Gluten Powder | 0.16 | 0.20 |
| Oat Protein Powder | 0.22 | 0.28 |
| Milk Protein Isolate | 0.14 | 0.18 |
| Dried Whole Eggs | 0.24 | 0.31 |
| Codfish Powder NM | 0.16 | 0.20 |
| Salmon Powder (Source 1) | 0.13 | — |
| Salmon Powder (Source 2) | — | 0.60 |
| Shrimp Powder | 0.19 | 0.23 |
| Sesame Seed Flour | 0.21 | 0.26 |
| Dry Vitamin D3 100 SD/S | 0.02 | 0.02 |
| Maltodextrin | 23.10 | 28.88 |
| Extra Fine Granulated Sugar | 7.70 | 9.63 |
| Water | 62.26 | 52.76 |
| Natural Juicy Orange Flavor WONF | 0.05 | — |
| #10757 (IFF Ottens Flavors) | | |
| Gellan Gum | 0.10 | 0.10 |
| Dipotassium Phosphate, Powder | 0.10 | 0.10 |
| Canola Oil | 1.00 | 1.00 |
| Totals | 100 | 100 |

Protocept A was processed without any issues, and resulted in a milky product with minimal settling out over time. In contrast, Protocept B's increased concentration correlated to a thick product prior to heat treatment. Table 2 compares the measured viscosities of Protocept A and Protocept B along with known reference products.

TABLE 2

| Product | Viscosity (cP, 70° F.) |
|---|---|
| Protocept A | 85 |
| Protocept B | 255 |
| Olive Oil (Reference Product) | ~80 |
| Maple Syrup (Reference Product) | ~300 |

As a result of Protocept B's increased viscosity, some air was entrapped during processing, and there was foam formed in the headspace when the product was filled into bottles. The increased viscosity also exerted considerable stress on the homogenizer, and the finished product was less uniform and less stable overall. Moreover, air entrapment in the U.H.T unit has the potential to compromise the lethality of the heat treatment, as air interferes with the conduction of heat from the unit into the product to kill pathogens. Additionally, the flavor addition in Protocept A did not result in a significant reduction in marine off-notes.

Furthermore, Protocepts A and B differed in salmon ingredients. Protocept A contained hydrolyzed salmon protein which did not pass the allergenicity test. This issue was alleviated by the replacement with a different salmon powder and adjustment of the formulation to deliver the minimum 30 mg of protein needed in both Protocepts B and C.

Testing of Protocepts A and B was used in the design of an optimized formulation and process flow. The optimized formulation (Protocept C) has a 25 g serving size, and was able to be processed without issues. This serving weight correlates to a 22.3 mL serving size, obtained by weighing 25 g of Protocept A in a 25 mL graduated cylinder and measuring the volumetric value at the meniscus. Protocept C did not include flavor, and included the adjusted amount of salmon powder. Table 3 shows the Protocept C formulation.

TABLE 3

| | % Total Formula |
|---|---|
| Ingredient | Protocept C |
| Peanut Flour | 0.25 |
| Soy Protein Isolate | 0.14 |
| Blanched Almond Flour | 0.56 |
| Cashew Flour | 0.67 |
| Hazelnut Flour | 0.41 |
| Pecan Meal | 1.33 |
| Pistachio Meal | 0.57 |
| Walnut Flour | 0.28 |
| Wheat Gluten Powder | 0.16 |
| Oat Protein Powder | 0.22 |
| Milk Protein Isolate | 0.14 |
| Dried Whole Eggs | 0.24 |
| Codfish Powder NM | 0.16 |
| Salmon Powder (Source 2) | 0.48 |
| Shrimp Powder | 0.19 |
| Sesame Seed Flour | 0.21 |
| Dry Vitamin D3 100 SD/S | 0.02 |
| Maltodextrin | 23.10 |
| Extra Fine Granulated Sugar | 7.70 |
| Water | 61.97 |
| Natural Juicy Orange Flavor WONF | — |
| #10757 (IFF Ottens Flavors) | |
| Gellan Gum | 0.10 |
| Dipotassium Phosphate, Powder | 0.10 |
| Canola Oil | 1.00 |
| Totals | 100 |

A proposed, optimized process flow is as follows. First, in a dry mill, 15 of the allergenic ingredients (which can exclude one ingredient, e.g., the peanut ingredient) in proper ratios for protein dosing along with bulking ingredients to lower moisture and fat content to acceptable levels as needed (maltodextrin and sugar) will be dry milled to achieve desired particle size. The excluded ingredient, e.g., the peanut ingredient, will be dry milled separately or a commercially sourced peanut ingredient with a granularity similar to the dry milled 15 allergenic ingredient blend will be used. Next, the dry milled 15 allergenic ingredient dry mix and the additional ingredient, e.g., the peanut ingredient, will be incorporated into liquid format by shear mixing into lukewarm water to solubilize and suspend the solids, and the product will be held in a tank for 1 hour with constant agitation to hydrate the proteins. Next, the shear mixed product will be wet milled/homogenized to reduce particle size in the liquid suspension or slurry, with recirculation of the product multiple times to decrease sedimentation and improve opacity. Following homogenization, the remaining ingredients will be incorporated by shear mixing in the fat, buffer, and gellan gum to improve stability, visual appearance and the suspension of the solids. Flavor will be added, if needed. Next, an ultra-high temperature unit will be used for ultra-high temperature processing for 15 seconds at 287° F. for a thermal lethality step to ensure food product safety. The resulting product will further homogenized with an in-line homogenizer to produce a consistent range of particle sizes for solid components and fat globules, and to uniformly distribute particles throughout the liquid matrix. Lastly, the composition will be aseptically filled into single-serve packing for storage at ambient temperature. A key difference in the optimized process flow is that the filling will be done under aseptic conditions, as opposed to clean fill. This will allow for the product to be stored at ambient conditions as a shelf stable product.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

What is claimed is:

1. A process for producing a homogenized liquid mixed allergen composition comprising:
dry blending a dry mixture, wherein the dry mixture comprises 6 to 20 allergens and a bulking agent;
milling the dry mixture and passing the milled dry mixture through a large screen to obtain a first pass mixture;
milling the first pass mixture and passing the first pass mixture through a small screen to obtain a fine particle mixture with substantially consistent particle size;
mixing the fine particle mixture into water to obtain a hydrated mixture; and
passing the hydrated mixture through a homogenizer to obtain a homogenized liquid mixed allergen composition.

2. The process of claim 1, wherein milling the dry mixture comprises using a rotor speed of about 7,500 RPM.

3. The process of claim 1 or 2, wherein milling the first pass mixture comprises using a rotor speed of about 7,500 RPM.

4. The process of any one of claims 1-3, wherein the large screen has an opening size of about 0.033 inches.

5. The process of any one of claims 1-4, wherein the small screen has an opening size of about 0.020 inches.

6. The process of any one of claims 1-5, wherein milling the dry mixture and/or milling the first pass mixture further comprises pulsing a vacuum suction through the mill.

7. The process of any one of claims 1-6, wherein the bulking agent comprises maltodextrin.

8. The process of claim 7, wherein the bulking agent further comprises sucrose.

9. The process of any one of claims 1-8, wherein the bulking agent comprises a weight ratio of about 3:1 maltodextrin and sucrose.

10. The process of any one of claims 1-9, wherein mixing the fine particle mixture into water comprises shear mixing.

11. The process of claim 10, further comprising hydrating the fine particle mixture in the water for about 1 hour or more.

12. The process of any one of claims 1-11, further comprising processing the liquid mixed allergen mixture at an ultra-high temperature.

13. The process of claim 12, wherein the ultra-high temperature is about 287° F. or higher.

14. The process of claim 12 or 13, wherein the processing of the liquid mixed allergen mixture at an ultra-high temperature occurs after passing the hydrated mixture through the homogenizer.

15. The process of any one of claims 1-14, further comprising shear mixing the homogenized liquid allergen mixture with one or more excipients to obtain a shear-mixed homogenized liquid allergen mixture.

16. The process of claim 15 wherein the one or more excipients are each selected from the group consisting of: a food safe oil, a polysaccharide, flavoring, and a food safe salt.

17. The process of claim 16, wherein the polysaccharide is gellan gum.

18. The process of claim 16 or 17, wherein the food safe salt is dipotassium phosphate.

19. The process of any one of claims 1-18, further comprising in-line homogenizing the shear-mixed homogenized liquid allergen mixture.

20. The process of any one of claims 1-19, wherein one or more additional allergens are dry milled separately and optionally mixed with the dry mixture, the first pass mixture, and/or the fine particle mixture.

21. A liquid mixed allergen composition comprising a homogenized liquid mixed allergen composition wherein the homogenized liquid mixed allergen composition is produced by a process of any one of claims 1-20.

22. A liquid homogenized mixed allergen composition comprising:
12 to 16 different protein allergens; maltodextrin; sucrose; oil, and optionally a vitamin, wherein the weight ratio of maltodextrin to sucrose is about 3:1.

23. The liquid homogenized mixed allergen composition of claim 22, wherein the vitamin is vitamin D.

24. The liquid homogenized mixed allergen composition of claim 22, further comprising gellan gum.

25. A unit dose of about 20 mL to about 30 mL of a liquid homogenized mixed allergen composition comprising: 12 to 16 different protein allergens; maltodextrin; sucrose; oil, and optionally a vitamin, wherein the weight ratio of maltodextrin to sucrose is about 3:1.
